(19) United States
(12) Patent Application Publication
(10) Pub. No.: US 2008/0233052 A1
(43) Pub. Date: Sep. 25, 2008

(54) Contrast Agents

(76) Inventor: Oskar Axelsson, Lomma (SE)

Correspondence Address: GE HEALTHCARE, INC., IP DEPARTMENT, 101 CARNEGIE CENTER, PRINCETON, NJ, 08540-6231, US

(21) Appl. No.: 11/913,079

(22) PCT Filed: May 19, 2006
(86) PCT No.: PCT/NO2006/000186

§ 371 Date: May 20, 2008

(30) Foreign Application Priority Data
May 20, 2005 (NO) 20052429

(51) Int. Cl. A61K 49/18; A61K 49/04; A61K 49/00

(52) U.S. Cl. 424/9.42; 424/9.1

(57) ABSTRACT

The present invention relates to particles comprising cores of tungsten or tungsten in mixture with other metallic elements wherein said cores are coated and have an average size of at least 20 nm. Further, the invention relates to pharmaceuticals containing such particles, and to the use of such pharmaceuticals specifically as contrast agents in diagnostic imaging, in particular in X-ray imaging of atherosclerotic plaque and liver tumours.

The present invention relates to particles susceptible for uptake by macrophages and/or Kupffer cells and to pharmaceuticals containing such particles. The particles comprise coated cores of the metallic element of tungsten or of tungsten in mixture with other metallic elements wherein the average diameter of said particle is greater than 20 nm. The invention also relates to the use of such pharmaceuticals as contrast agents in diagnostic imaging, in particular in X-ray imaging of atherosclerotic plaque and liver tumors, and to contrast media containing such cores of the metallic element of tungsten or tungsten in mixture with other metallic elements

All diagnostic imaging is based on the achievement of different signal levels from different structures within the body. Thus in X-ray imaging for example, for a given body structure to be visible in the image, the X-ray attenuation by that structure must differ from that of the surrounding tissues. The difference in signal between the body structure and its surroundings is frequently termed contrast and much effort has been devoted to means of enhancing contrast in diagnostic imaging since the greater the contrast between a body structure and its surroundings the higher the quality of the images and the greater their value to the physician performing the diagnosis. Moreover, the greater the contrast, the smaller the body structures that may be visualized in the imaging procedures. I.e. increased contrast can lead to increased spatial resolution and thereby achieving a safer detection of the target for the diagnostic procedure.

The diagnostic quality of images is, for a given spatial resolution, strongly dependent on the inherent noise level in the imaging procedure, and the ratio of the contrast level to the noise level can thus be seen to represent an effective diagnostic quality factor for diagnostic images. The ratio of the signal level and the noice level is usually denoted signal to noise ratio, abbreviated SNR.

Achieving improvement of the diagnostic quality factor has long been and still remains an important goal. In techniques such as X-ray, magnetic resonance imaging (MRI) and ultrasound, one approach to improve the diagnostic quality factor has been to introduce contrast enhancing materials, contrast agents, into the body region to be imaged.

Thus in X-ray for example early examples of contrast agents were insoluble inorganic barium salts which enhanced X-ray attenuation in the body zones into which they distributed. More recently the field of X-ray contrast agents has been dominated by soluble iodine containing compounds and specifically iodinated aryl compounds such as those marketed by Amersham Health AS under the trade names Omnipaque™ and Visipaque™.

Work on X-ray contrast agents having heavy metals as the contrast enhancing element has to a great extent concentrated on aminopolycarboxylic acid (APCA) chelates of heavy metal ions. Recognising that effective imaging of many body sites requires localization at the body sites in question of relatively high concentrations of the metal ions, there have been suggestions that polychelants, that is substances possessing more than one separate chelant moiety, might be used to achieve this. Further work has been concentrated on the use of multinuclear complexes that are complexes wherein the complexed moiety itself comprises two or more contrast enhancing atoms, see Yu, S. B. and Watson, A. D. in Chem. Rev. 1999, 2353-2377. Thus, for X-ray or ultrasound the complexes would comprise two or more heavy metal atoms and for MRI the complex would contain two or more metal atoms with paramagnetic properties. Yu, S. B. and Watson, A. D. also discuss use of metal-based X-ray contrast media. Tungsten powder is noted for use as an X-ray contrast additive in embolic agents used in the treatment and preoperative embolisation of hypervascular tumors. However, they find it likely that general intravascular use of heavy metal complexes is limited by safety concerns and dosage requirements.

X-ray contrast agents for parenteral administration are mainly hydrophilic of nature and have approximately the same extracellular biodistribution and are preferably renally excreted. Various attempts are made to achieve organ specific X-ray contrast agents that accumulate in organs and cells of the body and which can be administered parenterally. Iodinated aryl based X-ray contrast agent for example has been linked to macromolecular substrates such as starch in order to improve their vascular half-life. Potential liver contrast agents based on biodegradable particles are proposed in e.g. WO-A-8900988 and WO 9007491. Liposomes containing ionic or non-ionic iodinated aryl compounds have also been suggested, see e.g. WO-A-8809165 and U.S. Pat. No. 5,676,928. In the later years targeting moieties such as specific vectors binding to receptors at the target organs or cells have been proposed.

PCT/NO2004/00036 proposes particles with a core of the metallic element tungsten optionally together with other metallic elements and being coated with a coating layer. The particles should preferable be below the kidney threshold of about 6 to 7 nm to ensure excretion through the kidneys. The coating could be monomeric and polymeric and provide particles with a short half-life in vivo. Surface coatings with targeting moieties embedded, such as antibodies, are also proposed for the targeting of various body organs and structures, including tumours and macrophages.

Cardiology and oncology are important medical areas where there is a continuing need for reliable diagnosis of diseases and for monitoring the treatment of diseases.

Cardiovascular disease (CVD) is the leading course of death in the Western world and encompasses dysfunctional conditions of the heart, arteries, veins and lungs; which supply oxygen to vital life-sustaining areas of the body like the brain, the heart itself, and other vital organs. These conditions include coronary heart disease (CHD), coronary artery disease (CAD), chronic obstructive pulmonary disease (COPD), atherosclerosis, and thrombosis, and can lead to potentially life-threatening events as myocardial infarction (Ml), pulmonary embolism (PE) and stroke. One factor in common for all these diseases is the involvement of macrophages.

CHD is the most prevalent of the cardiovascular diseases. In 1998 it was estimated that CHD was the cause of 7 million deaths worldwide. CAD precedes CHD, and in the majority of cases the underlying cause is atherosclerosis. Atherosclerosis can be a benign disease for decades until the atherosclerotic plaque becomes atheromatous and potentially symptom producing. The plaque can obstruct blood flow resulting in stenosis of the artery, leading to acute myocardial ischemia in the case of coronary arteries. Additionally, mature atherosclerotic plaques can rupture resulting in the exposure of thrombogenic lipid, and these plaque components can form a trombous which completely blocks the artery. Angina is a common manifestation of CHD and is often the forerunner to more serious complications such as acute coronary syndromes including unstable angina, myocardial infarction and sudden cardiac death. Plaque rupture precedes the majority of clinical events and the vulnerability of plaque is the most important predictor of clinical outcome.

In cardiology, safe and early diagnosis of plaque and in particular of atherosclerotic plaque is therefore of great importance. Early diagnosis dramatically improves the outcome of the treatment of such diseases. It is well known that atherosclerotic plaques are infiltrated with a relatively large fraction of macrophages. The more vulnerable the plaque is the higher is the amount of macrophages in the plaque. A histological definition of “vulnerable plaque” is a plaque with a fibrous cap thinner than 65 μm and with a content of more that 25 cells in a 3.3 mm microscopic field which would correspond to an amount of about 4% macrophages in the fibrous cap, see “Handbook in vulnerable plaque”, Martin Dunitz; N.Y. Eds. R. Waksman and P. W. Serruys, pp 39-41. Macrophages are usually of a size between 8 and 30 μm. Macrophages will recognise and take up particles by phagocytosis from the blood pool. Macrophages can hence be used as a tool to concentrate or target contrast agents to specific macrophage containing organs or structures in the body.

In oncology, liver tumours such as hepatomas and metastatic spread to the liver are major causes of death in the world. There is a continuing need for methods and products to help in the early diagnosis of cancer. Cancer tissues in general have different vascularity from healthy tissues and may be detected as an area of modified contrast. However, X-ray examination of the liver will typically require high amounts of iodinated contrast agent and injection of contrast agent containing ca. 9 g iodine will be required, see WO-A-8809165. The Kupffer cells reside in the liver and will take up and initially break down particles in a similar fashion as the macrophages. Kupffer cells are not present or only present to a low extent in liver tumour tissue. Hence there is a possibility to identify cancerous liver tissue as tissue that give no or very low signal in X-ray examination of the liver after administration of a suitable X-ray contrast agent.

None of the attempts to provide specific X-ray contrast agents for imaging of atherosclerotic plaque and/or liver tumours has resulted in commercial products. Problems encountered in this regard has been insufficient contrast in the target organ or structure and the need for very high doses of contrast agents e.g. in the form of X-ray contrast agents enclosed in liposomes which may lead to adverse reactions. It has hence been difficult to achieve a satisfactory signal to noise ratio (SNR) sufficient to secure a safe and accurate diagnosis in particular of small lesions.

It has now surprisingly been found that compounds being susceptible for uptake by macrophages and/or Kupffer cells can be provided that provide sufficient contrast in X-ray contrast examination of the vascular bed and for the identification of tumour tissue. Such compounds are particles comprising a core of the metallic element tungsten optionally together with other metallic elements and being coated with a coating layer wherein the average diameter of said particle is greater than 20 nm.

The invention will now be described in further details. The various embodiments are also specified in the attached claims and form part of the entire description of the invention.

Coated nanoparticles comprising tungsten are enclosed in PCT/NO2004/00036, which is hereby incorporated by reference. The particles of this document are however small to facilitate fast excretion; preferably their size is below the kidney threshold of 6 to 7 nm to secure secretion through the kidneys.

According to the present invention it has been found that in order to achieve sufficient uptake by the macrophages and/or Kupffer cells and to achieve sufficient contrast and SNR, it is necessary to provide particles of larger sizes than previously suggested, in particular particles with an average diameter of at least 20 nm.

It should be noted that the terms core, metallic core and tungsten core are used interchangeably in the further document. By the expression pharmaceuticals is also enclosed the particles which constitute the active principle of the pharmaceutical. Further embodiments are specified in the attached claims and will be outlined in the text.

The compounds of the invention are particles comprising a core and a coating layer. The particle size can vary in range but should be at least 20 nm, e.g. from 20 to 1000 nm and preferably from 20 to 200 nm. Even more preferably the particle size should be from about 100 to 200 nm. The particle size should therefore preferably be above the kidney threshold of about 6 to 7 nm (Kobayashi, H.; Brechbiel, M. W. Molecular Imaging 2, 1 (2003)).

Metallic tungsten has a relatively high X-ray attenuation value, a low toxicity and is available at an acceptable price.

The core of the particle contains tungsten in its metallic form or tungsten in mixture with other suitable metallic elements. Preferably the tungsten content is between 20 and 100 weight %, more preferably between 50 and 100 weight %, and even more preferably of 85 to 100 weight % and particularly preferably between 95 and 100 weight %. Cores of about 100% tungsten are generally preferred.

Introducing other metallic elements in the tungsten core can provide improved properties to the core e.g., can improve the stability, monodispersity, the synthesis and/or the rate of formation of the metal core. Preferably 5 to 15 weight % of rhenium, iridium, niobium, tantalum or molybdenum either as a single element or as mixtures of elements are feasible additives, most preferred are rhenium and iridium. All these elements are miscible with tungsten and small amounts of rhenium and/or iridium improve the low temperature plasticity of the metallic core.

It is important that the metallic cores which provide the attenuating properties to the particles are of a sufficient size with regard to this property taking into consideration the preferred total size of the particle. The particle must hence contain an as high amount as possible of metal atoms to provide the desired attenuating properties. The particle must also be larger than the kidney threshold to avoid fast kidney excretion and to keep the particles in the blood pool long enough to fascilitate uptake by the macrophages and/or Kupffer cells. Macrophages and Kupffer cells are known to be able to take up particles of a size from 20 nm to 1 μm (1000 nm). The upper size of the particles should be small enough so that the particles can be injected as a uniform solution not containing particles of a size that will clog the capillaries, particularly the lung capillaries and the maximum size should therefore be below about 8 μm and preferably the average size of the particles should be 1 μm or below.

Since the tungsten containing core is reactive to a greater or lesser extent, the metallic core must be coated in order to passivate the reactive surface. The properties of the coating should provide a protection to the metallic core such that the core does not react e.g. ignite when exposed to air, or react when formulated for in vivo use or react in the in vivo environment. Preferably the coating should maintain its properties until the particles are excreted from the body to which they are administered to such degree that the tungsten surface of the core does not become reactive. The coating should also provide particles that facilitate uptake by macrophages and/or Kupffer cells. It is also important that the coating is such that the particles have a low tendency to form aggregates, particularly in vivo. At the same time the coating must be relatively thin in order that for a given particle size the amount of contrast enhancing material can be optimized. The thickness of the coating should preferably be below 50 nm, more preferred below 20 nm and even more preferred between 1 and 5 nm. The binding between the metal core and the coating should also be sufficiently strong to avoid disintegration between the metallic core and the coating.

Although it is preferred that the coating remains intact throughout the residence time of the particles in the body, some leakage of the core metals such as tungsten is acceptable since tungsten is of relatively low toxicity and released metal will rapidly be excreted through the kidneys.

The water solubility of the nanoparticles must be sufficiently high when the pharmaceutical is formulated for parenteral administration, e.g. for injection into a vein or an artery. The coating should therefore be chosen such that it contributes to the solubility of the particles. Viscosity and osmolality of the particles in solution must also be taken into account when choosing the coating. The viscosity and the osmolality should be as low as possible to provide ease of administration and to avoid adverse effects in particular adverse effects connected to the osmolality. The solution of the particles for administration should be slightly hypertonic or isotonic.

The coating layer should preferably be a polymeric coating layer. The polymeric coating layer comprises a layer of any polymeric material suitable for pharmaceutical use containing a minimum number of negatively charged groups per particle to promote recognition of the particles by the macrophages and/or Kupffer cells. The coating should cover the tungsten surface densely enough to passivate it. The polymeric surface layer can be covalently bound to the metallic core surface or adsorbed and held by non-covalent forces. A polymeric coating winds across the metal surface and interacts with the metal core at multiple points along the chain. The efficacy of the particles depends on that the tungsten core of the particles constitutes the highest possible fraction of the particle. It is preferred that the coating layer is as thin as possible and at the same time provides the necessary passivation of the tungsten core surface. The polymer can be a natural or synthetic homopolymer or copolymer. Numerous polymers are available for the purpose and the skilled artisan will be able to choose suitable polymers known from the state of art. Useful classes of polymers include polyethers (e.g. PEG and optionally branched), polyacetals, polyvinylalchohols and polar derivatives thereof, polyesters, polycarbonates, polyamides including aliphatic and aromatic polyamides and polypeptides, classes of carbohydrates such as starch and cellulose, polycyanoacrylates and polycyanometacrylates, preferably provided that the polymers include a minimum of negatively charged groups and most preferable also contains a fraction of lipophilic groups as well as hydrophilic groups in order to promote solubility.

To further enhance the recognition of the particles by the macrophages and/or the Kupffer cells, the particles should preferably comprise a metal core coated by a negatively charged coating layer in the form of chemical entities with negative charged groups. The number of charges will depend upon the size of the metallic core and also the size of the coated nanoparticle. Coatings comprising charged groups will provide particles that repel each other when in solution, and formation of particle clusters is thereby substantially or partially avoided. Avoiding formation of clusters of the coated particles enhance the solubility of the particles. Further, the viscosity of the particle formulation will be kept in a preferred range.

On the other hand the formulation of charged particles will comprise neutralising counter ions and this will lead to a rise in the osmolality. However, since the nanoparticles contain a large number of tungsten atoms it is possible to achieve solutions that contain a sufficient concentration with respect to tungsten atoms with an acceptable osmolality.

The charged groups must be in their ionic form at the pH of the environment where the compound is used. Most importantly they must be in charged form at physiological pH, in particular at the pH of blood.

Anionic groups exerting negative charges can be a wide variety of groups known to the skilled artisan. Of particular importance are acidic groups such as carboxylic acid groups, sulphonic acid groups, phosphoric acids groups and also acidic heterocyclic groups such as tetrazoles or 5-hydroxyisooxazoles.

The monomers of the coating material should preferably further comprise lipophilic groups in the form of at least a fraction of lipophilic groups in the polymeric coating layer. Lipophilicity enhances the recognition of the macrophages and/or the Kuppfer cells of the particles and enhances the uptake by the target cells. However, it is important to balance the lipophilicity of the coating layer with the water solubility of the particles, hence the coating layer of non-metallic material should also comprise at least a fraction of molecules that are hydrophilic.

The lipophilic groups can be introduced into the polymeric coating as a lipophilic monomer. One example of suitable lipophilic monomer groups is alkylacrylates wherein the alkyl groups are straight or branched alkyl groups of at least 8 carbon atoms. A particularly preferred alkylacrylate is dodecyl acrylate.

Polymers made of acrylic acid monomers are specifically preferred. In order to obtain a layer with a controlled and suitable number of charged groups, copolymers are also preferred wherein the copolymer can contain 2 or more momomeric entities or blocks. At least one of the monomers shall provide negatively charged groups to the polymer coating. The negative charged coatings promote recognition of the particles by the macrophages and/or Kupffer cells as noted above and also increase the water-solubility and reduce the risk of particle aggregation. However, the charged coating layer also increases the osmolality of the particles. Thus, the number of charge carrying groups should be kept at a minimum. In preparations, a neutral monomer combined with a charged monomer in molar ratios below 20:1, preferably from 10:1 to 10:1.5 can provide a polymer with a suitable number of charges for the particles. Possibly, this ratio could be increased even further.

Examples of suitable monomers to be used to form the polymeric coating are:

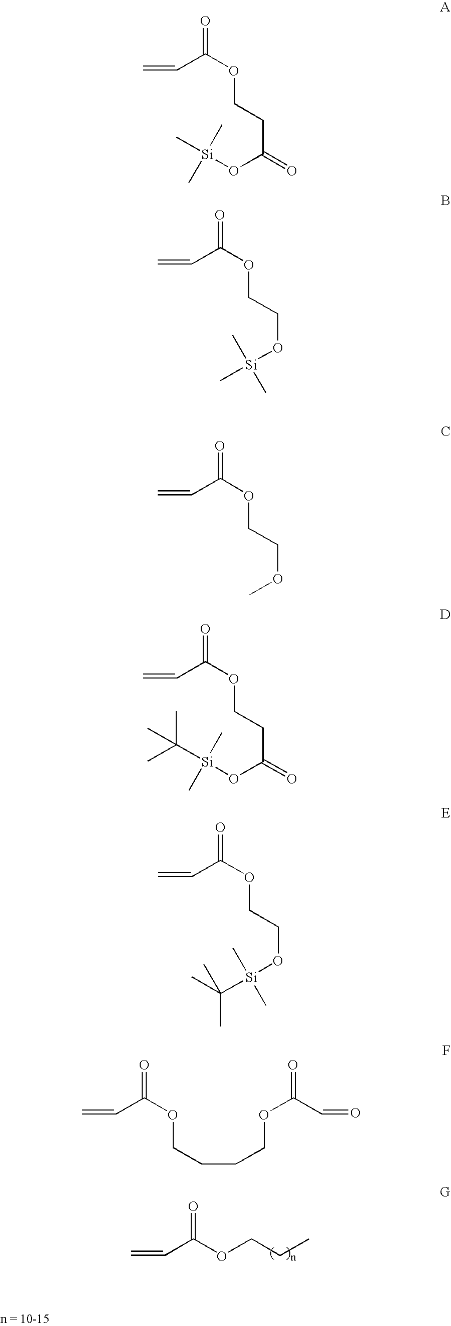

Use of monomer F forms a cross-linked polymer.

Generally, the polymer coated particles are prepared by thermally decomposing a source of tungsten (0), e.g. tungsten hexacarbonyl, W(CO)₆, in a high-boiling, dried and deoxygenated solvent in the presence of one or more of the monomers. A thermally induced polymerization of the monomers takes place, covering the tungsten particles formed from the decomposition, with a polymeric coating. When the monomers comprise silylether-protected polar groups (—OH, —COOH) the protecting groups are cleaved in aqueous solution to yield the hydrophilic polymer coated particles.

Dry solvents should generally be used. Hygroscopic solvents (diglyme, triglyme) should be percolated through alumina and stored over molecular sieves. All solvents should be deoxygenated by letting a stream of argon bubble through the solvent for 25-30 minutes before they are used in the reactions. The choice of solvent for this process is critical since there are several criteria to be fulfilled. One is the ability to dissolve the starting materials and at the same time keep the final polymer coated particles in solution. The polyethers di- and triglyme are particularly useful here. The high boiling point of tetraglyme in particular, will allow the temperature to reach the level where the last carbon monoxide molecules leave the particles. Other useful solvents would be diphenyl ether and other inert high-boiling aromatic compounds. Also trioctyl phosphine oxide (and other alkyl analogs), trioctyl phosphine (and other alkyl analogs), high boiling amides and esters would be useful.

Another important process parameter is the ability to control the tendency of W(CO)₆ to sublimate out of the reaction mixture. This can be achieved by mixing in a small fraction of a lower boiling solvent to continuously wash back any solid tungsten hexacarbonyl from the condenser or vessel walls. Cyclooctane and n-heptane would be good choices when used in 5 to 15% volume fractions.

For the work-up of the particles, precipitation by the addition of pentane or other low-boiling alkanes would be convenient. A low boiling point solvent is advantageous when the particles are to be dried.

The preparation and work-up procedures are further described in the specific examples.

Contrast agents are frequently administered parentally, e.g. intravenously, intra-arterially or subcutaneously. Contrast agents can also be administered orally or via an external duct, e.g. to the gastrointestinal tract, the bladder or the uterus. Suitable carriers are well known in the art and will vary depending on e.g. the administration route. The choice of carriers such as excipients or solvents is within the ability of the skilled artisan. Usually aqueous carriers are used for dissolving or suspending the pharmaceutical, e.g. to produce a contrast agent. Various aqueous carriers may be used such as water, buffered water, saline, glycine, hyaluronic acid and the like.

In one embodiment the invention provides pharmaceuticals comprising the particles as hereinbefore described together with a pharmaceutically acceptable solvent or excipient, and specifically such pharmaceuticals for use as contrast agents, in particular for use as X-ray contrast agents.

It will be possible to formulate solutions containing the particles of the invention having from about 1.0 to about 4.5 g tungsten/ml solution, more specifically from 1.5 to about 3.0 g tungsten/ml water and most specifically about 2.2 g tungsten/ml water.

For use as pharmaceuticals the tungsten containing particles must be sterilized, this can be done by techniques well known is the state of art. The particles can be provided in a sterile solution or dispersion or alternatively in dry form, e.g. in lyophilized form.

In further embodiments the invention provides a method of diagnosis and a method of imaging where the particles are administered to a human or animal body. The body is examined with a diagnostic device and data are compiled from the examination. The data can be further processed if needed to facilitate that the data can be used to create an image and to reach to a diagnosis. The data can be used for the visualisation and identification of plaque, specifically atherosclerotic plaque and more specifically vulnerable atherosclerotic plaque or for the visualisation and identification of liver tumours.

As noted above, plaques contain macrophages and vulnerable atherosclerotic plaque contain a high amount of macrophages. Plaque can therefore be diagnosed as areas with a high uptake of the contrast agents according to the invention. For diagnosis of plaque it is preferred to administer the contrast agent of the invention by infusion, preferably a relatively slow infusion of a diluted contrast agent preparation.

For the diagnosis of liver tumours these are identified as areas in the liver that are not enhanced by the contrast agent. This is because the contrast agents are taken up by the liver Kupffer cells. Kupffer cells are not present or only present to a very low extent in liver tumour tissue. Active uptake of the particles of the diagnostic agents of the invention by the Kupffer cells would then make it possible to identify liver tumours as areas of the liver that is not contrast enhanced. For liver tumour diagnosis, it is preferred to administer the diagnostic agent of the invention as a single bolus dose.

Although if may be possible to use plain X-ray imaging in the signal uptake in the methods of the invention at least for large lesions, X-ray Computed Tomography (CT) is highly preferred. The molar extinction of tungsten for CT relevant X-rays (120 kV) is 5500 Hounsfield units/mol (HU). A modern CT has a noise level of about 15 HU for a reasonable resolution, and it is possible to identify lesions of less than 1 mm². In the not too distant future with CT having an enhanced resolution it should be possible to identify contrast enhanced plaque smaller than 1 mm², and to differentiate between large weakly inflamed plaque and small highly inflamed plaque.

Fast CT signal uptake is important, particularly for imaging of atherosclerotic plaque. A heart scan can be done in 100 ms and if synchronized with the heart beat, a motion free image can be acquired in the diastolic phase of a single heart beat. Multiple acquisitions for averaging can also be made, however alignment problems of very small lesions may cause loss on SNR.

The invention will hereinafter be further illustrated with the non-limiting examples. All temperatures are in ° C. The tungsten content in the particles was determined by X-ray fluorescence spectroscopy. Dynamic Light Scattering was used to determine particle size of one of the preparations.

### EXAMPLES

The following monomers were used in the Examples:

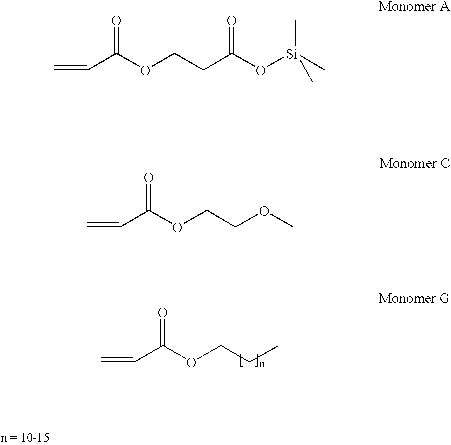

### Example 1

### Preparation of Tungsten Nano-Particles in the Size Range of 20-200 nm

Tetraglyme (100 ml), dried and deairated, was put in a three necked flask, equipped with a reflux condenser, a thermostat and a magnetic stirrer. A mixture of monomer C (1.17 g, 9.0 mmol) and monomer A (0.36 g, 1.6 mmol) and W(CO)₆ (1.5 g, 4.3 mmol) was added to the solvent and a stream of argon was bubbled through the mixture for 10 minutes. The glassware set-up was sealed with a septum at the top of the condenser and an argon atmosphere was introduced by three consecutive vacuum/argon cycles, performed through a needle in the septum. The reaction mixture was stirred for 3 h at 150° C. after which the temperature was raised to 220° C. and the mixture was stirred at this temperature for an additional hour. The black reaction mixture was then cooled to room temperature and the particles were collected by centrifuging at 3000 g for 15 minutes. The black precipitation was washed with pentane twice and dried in vacuum. Yield: 400 mg dark powder. Tungsten content (X-ray fluorescence spectroscopy): 65-70%. Size distribution (Dynamic Light Scattering): 20-200 nm.

The initially water-insoluble particles were made water-soluble by stirring them in water and titrating the slurry/solution to pH 7 with NaOH. The water-soluble particles were then recovered by freeze-drying.

### Example 2

### Preparation of Tungsten Nano-Particles with a Coating with Lipophilic Groups in the Size Range of 20-200 nm

The preparation described in Example 1 is modified by adding a lipophilic monomer (e.g. monomer G) to the initial reaction mixture. The amount of the lipophilic monomer is kept at a level low enough not to interfere with the water-solubility of the particles. Preparation and work-up procedures are preformed as in Example 1.

1. A particle characterized in comprising a core of the metallic element tungsten optionally together with other metallic elements, and being coated with a coating layer, and wherein the average diameter of said particle is at least 20 nm.

2. A particle as claimed in claim 1 of a diameter in the range of at least 20 nm to 1000nm.

3. (canceled)

4. A particle as claimed in claim 1 of a diameter in the range of 100 nm to 200 nm.

5. A particle as claimed in claim 1 wherein the core of the particle has a tungsten content of 20 to 100 weight % of metallic tungsten.

6. (canceled)

7. (canceled)

8. A particle as claimed in claim 1 wherein the core of the particle has a tungsten content of 95 to 100 weight % of metallic tungsten.

9. (canceled)

10. A particle as claimed in claim 1 wherein the core of the particle comprises metallic tungsten and one or more of the elements rhenium, iridium, niobium, tantalum or molybdenum in their metallic form.

11. A particle as claimed in claim 1 wherein the coating layer comprises a polymeric coating layer.

12. A particle as claimed in claim 1 wherein the coating layer comprises a negatively charged polymeric coating layer.

13. (canceled)

14. A particle as claimed in claim 1 wherein the coating layer provides the net negative charge from acidic groups such as carboxylic acid groups, sulphonic acid groups, phosphoric acid groups and acidic heterocyclic groups.

15. A particle as claimed in claim 11 wherein the polymeric coating layer comprises a hydrophilic polymer.

16. A particle as claimed in claim 11 wherein the polymeric coating layer comprises a homopolymer or a copolymer.

17. (canceled)

18. A particle as claimed in claim 11 wherein the polymeric coating layer comprises lipophilic groups.

19. A particle as claimed in claim 11 wherein the polymeric coating layer is formed from acrylic acid monomers.

20. (canceled)

21. A particle as claimed in claim 11 wherein the polymer comprises at least one lipophilic monomer.

22. A particle as claimed in claim 21 wherein the lipophilic monomer comprises alkylacrylates wherein the alkyl groups are straight or branched alkyl groups of at least 8 carbon atoms.

23. A particle as claimed in claim 22 wherein the alkylacrylates comprises dodecyl acrylate.

24. (canceled)

25. A particle as claimed in claim 1 characterized in being susceptible for uptake by macrophages.

26. (canceled)

27. A contrast agent comprising particles of claim 1 optionally together with a solvent or excipient.

28. A contrast agent as claimed in claim 1 being an X-ray contrast agent.

29. (canceled)

30. (canceled)

31. (canceled)

32. (canceled)

33. (canceled)

34. (canceled)

35. A method of diagnosis comprising administration of particles of claim 1 to a human or animal body, examining the body with a diagnostic device and compiling data from the examination.

36. A method of claim 35 wherein atherosclerotic plaque, particularly vulnerable atherosclerotic plaque or liver tumors are diagnosed.

37. (canceled)

38. (canceled)

39. (canceled)

40. A method of imaging, specifically X-ray imaging comprising administration of particles of claim 1 to a human or animal body, imaging the body with an imaging device, compiling data from the examination and optionally analysing the data.

41. A method of claim 40 wherein X-ray imaging comprises X-ray Computer Tomography imaging.

42. (canceled)

43. A process for the preparation of particles of claim 1 comprising decomposing a source of tungsten (0) in a high boiling, dried and deoxygenated solvent in the presence of one or more monomers and thereby effecting a thermally induced polymerization of the monomers.

44. A process as claimed in claim 43 wherein the source of tungsten (0) is tungsten hexacarbonyl (W(CO)₆).
